(19) United States
(12) Patent Application Publication
(10) Pub. No.: US 2024/0299620 A1
(43) Pub. Date: Sep. 12, 2024

(54) BIOENERGETIC-ACTIVE MATERIAL AND USE THEREOF

(71) Applicant: SHENZHEN INSTITUTES OF ADVANCED TECHNOLOGY, Guangdong (CN)

(72) Inventors: Jian LI, Guangdong (CN); Peng ZHANG, Guangdong (CN)

(73) Assignee: SHENZHEN INSTITUTES OF ADVANCED TECHNOLOGY, Guangdong (CN)

(21) Appl. No.: 18/574,727

(22) PCT Filed: Aug. 1, 2022
(86) PCT No.: PCT/CN2022/109458

§ 371 Date: Dec. 27, 2023

(30) Foreign Application Priority Data
Jul. 30, 2021 (CN) 202110875054.2

(51) Int. Cl. A61L 27/18; A61L 27/56

(52) U.S. Cl. CPC A61L 27/18; A61L 27/56; A61L 2430/02

(57) ABSTRACT

A bioenergetic-active material and use thereof are provided, and in particular to a bioenergetic-active material is a biodegradable polymer. A degradation product of the bioenergetic-active material is a metabolic intermediate via a tricarboxylic acid cycle and/or a glycolysis pathway, or a polymer monomer capable of being converted into a metabolic intermediate via a tricarboxylic acid cycle and/or a glycolysis pathway, or a polymer monomer capable of being converted into acetyl-coenzyme A. The degradation product of the bioenergetic-active material provides bioenergy for tissue cells via a tricarboxylic acid metabolic cycle or a glycolysis pathway, so that the problem that the traditional biodegradable material cannot continuously improve the stability of ATP in cells and the activity of related biomass is solved, and the bioenergetic-active material has a wide application prospect in the field of bone tissue regeneration, particularly in the aspect of large-sized bone defect repair.

### TECHNICAL FIELD

The present invention belongs to biomedical tissue engineering, and particularly relates to a bioenergetic-active material and use thereof.

### DESCRIPTION OF RELATED ART

Clinically, various types of bone defects caused by trauma, infection, bone tumor resection and the like are very common. There are 2.2 million bone graft patients worldwide every year [J. Van der Stok, E. M. Van Lieshout, Y. El-Massoudi, G. H. Van Kralingen, P. Patka, Bone substitutes in the Netherlands-a systematic literature review, Acta Biomater 7(2) (2011) 739-50]. According to the statistics from the American Academy of Orthopaedic Surgeons (AAOS), 6.3 million people are subjected to bone fractures in the United States every year, with a half million patients needing to receive bone grafts, with annual costs in bone fracture treatment alone of up to 200 billion dollars [D. C. Lobb, B. R. DeGeorge, Jr., A. B. Chhabra, Bone Graft Substitutes: Current Concepts and Future Expectations, J Hand Surg Am 44(6) (2019) 497-505 e2]. In China, over 3.5 million patients suffer from bone injury every year, and hundreds of thousands of new cases are added every year. The annual growth rate of hospitalization due to trauma is 7.2%, ranking second in the number of inpatients. The high incidence of bone defects makes bone grafts the most demanded medical consumables next to blood transfusion [G. H. Brundtland, A WHO Scientific Group on the Burden of Musculoskeletal Conditions at the Start of the New Millennium met in Geneva from 13 to 15 Jan. 2000., Who Tech Rep Ser 919 (2003) 1-218], which brings a heavy medical burden to society.

Autogenous bone grafting may be a good way for bone repair, but “limited donor” limits the extensive use thereof. The biodegradable polymer material has excellent biocompatibility and degradability, and thus is widely used in the research of bone tissue engineering. The biodegradable polymer materials include natural biodegradable polymers (such as collagen and chitosan) and synthetic biodegradable polymers (such as PLA, PLGA, and PCL), which are commonly-used bone tissue engineering scaffold materials at present due to their good biocompatibility and degradability [F. Asghari, M. Samiei, K. Adibkia, A. Akbarzadeh, S. Davaran, Biodegradable and biocompatible polymers for tissue engineering application: a review, Artif Cells Nanomed Biotechnol 45(2) (2017) 185-192]. Although the biodegradable polymer has a certain bone defect repair function, a series of defects that the degradation rate is uncontrollable, the mechanical property is poor, the acidic degradation product causes inflammatory reaction and the like are difficult to overcome still exist [Y. X. Lai, Y. Li, H. J. Cao, J. Long, X. L. Wang, L. Li, C. R. Li, Q. Y. Jia, B. Teng, T. T. Tang, J. Peng, D. Eglin, M. Alini, D. W. Grijpma, G. Richards, L. Qin, Osteogenic magnesium incorporated into PLGA/TCP porous scaffold by 3D printing for repairing challenging bone defect, Biomaterials 197 (2019) 207-219], which hinders the wide clinical application thereof.

The bone regeneration process is an energy-consuming process, and thus cellular energy metabolism plays a crucial role in tissue repair and regeneration. Adenosine triphosphate (ATP) is a major source of cellular energy and plays a role in many biological processes, including proliferation, migration and differentiation of cells [I. Gadjanski, S. Yodmuang, K. Spiller, S. Bhumiratana, G. Vunjak-Novakovic, Supplementation of Exogenous Adenosine 5-Triphosphate Enhances Mechanical Properties of 3D Cell-Agarose Constructs for Cartilage Tissue Engineering, Tissue Eng Pt A 19(19-20) (2013) 2188-2200]. Studies have shown that bioenergy has been successful as a potential therapeutic approach for regenerating in-vitro models or relatively-thin superficial tissues (e.g., skin). So far, 3D scaffolds with long-term bioenergy release effect for complex bone tissue defect repair have been rarely reported. This is due in large part to the fact that the use of existing biological scaffold materials cannot continuously improve the stability of ATP in cells and the activity of related biomass, which hinders the application and development of bioenergetic-active materials in bone tissue engineering.

### SUMMARY

In order to solve the defects and shortcomings in the prior art, the present invention provides a bioenergetic-active material and use thereof. The specific solution is as follows:

In a first aspect, the present invention provides a bioenergetic-active material, wherein the bioenergetic-active material is a biodegradable polymer; a degradation product of the bioenergetic-active material is a metabolic intermediate via a tricarboxylic acid cycle and/or a glycolysis pathway;
or a degradation product of the bioenergetic-active material is a polymeric monomer capable of being converted into a metabolic intermediate via a tricarboxylic acid cycle and/or a glycolysis pathway;
or a degradation product of the bioenergetic-active material is a polymer monomer capable of being converted into acetyl-coenzyme A.

Furthermore, in the above technical solution of the present invention, the metabolic intermediate of the tricarboxylic acid cycle comprises one or more of citrate, aconitase, isocitrate, oxalosuccinate, α-ketoglutarate, succinyl-coenzyme A, succinate, fumarate, malate, and adenosine triphosphate;
the metabolic intermediate of the glycolysis pathway comprises one or more of glucose-6-phosphate, fructose-6-phosphate, fructose-1,6-diphosphate, 3-phosphoglyceraldehyde, dihydroxyacetone phosphate, 1,3-diphosphoglycerate, 3-phosphoglycerate, 2-phosphoglycerate, phosphoenolpyruvate (PEP), and pyruvate;
the polymer monomer capable of being converted into acetyl-coenzyme A is 3-hydroxybutyric acid. 3-hydroxybutyric acid generates acetoacetate under the action of 3-hydroxybutyrate dehydrogenase, then acetoacetate and succinyl-coenzyme A (succinyl-CoA) are synthesized to obtain acetoacetyl-CoA under the action of 3-oxoacid CoA-transferase, and acetoacetyl-CoA reacts with a CoA under the action of acetyl-CoA C-acetyltransferases to obtain two acetyl-CoA, followed by entering into a tricarboxylic acid cycle to provide bioenergy for histiocytes.

Preferably, in the above technical solution of the present invention, the bioenergetic-active material is a polyhydroxyalkanoate having a degradation product of 3-hydroxybutyric acid.

Furthermore, the polyhydroxyalkanoate comprises one or more of poly(3-hydroxybutyrate-co-4-hydroxybutyrate) (P34HB), poly-3-hydroxybutyrate (PHB), poly(3-hydroxybutyrate-co-3-hydroxyvalerate) (PHBV), and poly(3-hydroxybutyrate-co-3-hydroxyhexanoate) (PHBHHx).

In a second aspect, the present invention provides use of the bioenergetic-active material in the field of bone tissue regeneration and repair.

In a third aspect, the present invention provides use of the bioenergetic-active material in the manufacture of a porous scaffold for bone tissue repair.

In a fourth aspect, the present invention provides a porous scaffold for bone tissue repair manufactured from the bioenergetic-active material. Furthermore, the porous scaffold for bone tissue repair may be manufactured by a conventional manufacturing method, or may be manufactured by a 3D printing technology.

In a fifth aspect, the present invention provides a method for manufacturing a 3D porous scaffold for bone tissue repair, which comprises the following steps:
(1) synthesizing the bioenergetic-active material; and
(2) manufacturing a 3D porous scaffold for bone tissue repair in combination with a 3D printing technology.

Furthermore, in the above method of the present invention, the bioenergetic-active material may be synthesized by a microbiological or chemical synthesis method, or may be synthesized by other methods. For example, poly(3-hydroxybutyrate-co-4-hydroxybutyrate) may be produced by fermentation of microorganism halophilic monads.

A 3D porous scaffold for bone tissue repair is manufactured by the method according to the fifth aspect of the present invention.

In a sixth aspect, the present invention provides use of a polyhydroxyalkanoate having a degradation product comprising 3-hydroxybutyric acid as a bioenergetic-active material with both bone tissue regeneration and angiogenesis functions, wherein 3-hydroxybutyric acid is, in a form of citrate, involved in bone formation via a tricarboxylic acid metabolic cycle, and 3-hydroxybutyric acid induces angiogenesis.

Furthermore, the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

In a seventh aspect, the present invention provides use of a polyhydroxyalkanoate having a degradation product comprising 3-hydroxybutyric acid in the preparation of a vascularized bone regeneration material.

Furthermore, the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

In an eighth aspect, the present invention provides use of a polyhydroxyalkanoate having a degradation product comprising 3-hydroxybutyric acid in the preparation of a large-sized bone defect repair material or a critical bone defect repair material.

Furthermore, the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

In a ninth aspect, the present invention provides a vascularized bone regeneration material, a large-sized bone defect repair material or a critical bone defect repair material, which is prepared from a polyhydroxyalkanoate having a degradation product comprising 3-hydroxybutyric acid.

Furthermore, the material is a porous scaffold manufactured from the polyhydroxyalkanoate having the degradation product comprising 3-hydroxybutyric acid.

Furthermore, the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

The present invention has the following beneficial effects:
1. The degradation product of the bioenergetic-active material provided in the present invention is a metabolic intermediate via a tricarboxylic acid cycle and/or glycolytic pathway, or a polymer monomer capable of being converted into a metabolic intermediate via a tricarboxylic acid cycle and/or glycolytic pathway, or a polymer monomer capable of being converted into acetyl-coenzyme A. The degradation product of the bioenergetic-active material provides biological energy for tissue cells via a tricarboxylic acid metabolic cycle or a glycolysis pathway, so that the problem that the traditional biodegradable material cannot continuously improve the stability of ATP in cells and the activity of related biomass is solved, and the bioenergetic-active material has a wide application prospect in the field of bone tissue regeneration, particularly in the aspect of large-sized bone defect repair.
2. The bioenergetic-active material poly(3-hydroxybutyrate-co-4-hydroxybutyrate) (P34HB) provided in the present invention is a degradable polymer polyester, and has unique bioenergy activity, mechanical property, biodegradability and biocompatibility, a main product of which 3-hydroxybutyric acid (3HB) generated by degradation is one of main components of ketone bodies in mammals, with no toxic effect on organisms, and can be used as an energy substance to promote the adhesion, proliferation and differentiation of cells. In addition, compared with polylactic acid, the poly(3-hydroxybutyrate-co-4-hydroxybutyrate) material can maintain a longer degradation time under the same condition. A longer degradation time of 6-12 months can be realized by adjustment of the proportion of 3-hydroxybutyric acid in the material according to the requirement of tissue repair. The poly(3-hydroxybutyrate-co-4-hydroxybutyrate) material can solve the problems that the traditional biodegradable materials cannot continuously improve the stability of ATP in cells and the activity of related biomass, and that an acidic degradation product causes inflammatory reaction, and thus is an excellent active scaffold material for bone repair with bioenergy activity and a bone formation promoting function.
3. The porous scaffold for bone tissue repair provided in the present invention is based on a bioenergetic-active material and a 3D printing technology, is controllable in structure, and has good biocompatibility, biodegradability and mechanical properties. The scaffold, after being implanted into a body, continuously degrades to generate a bioenergetic-active substance, and the bioenergetic-active substance can promote the proliferation, differentiation and mineralization of human bone mesenchymal stem cells (hBMSCs) and has a bone formation promoting function that conventional polymer scaffold materials do not have.
4. The 3D porous scaffold for bone tissue repair provided in the present invention is manufactured from the bioenergetic-active material poly(3-hydroxybutyrate-co-4-hydroxybutyrate) (P34HB) by a 3D printing technology, is controllable in structure, and has good biocompatibility, biodegradability and mechanical properties. The 3D porous scaffold, after being implanted into a body, continuously degrades to generate a bioenergetic-active substance 3-hydroxybutyric acid (3HB), and after the 3HB enters into a tricarboxylic acid metabolic cycle, it provides bioenergy for histiocytes and promotes the proliferation, differentiation and mineralization of human bone mesenchymal stem cells (hBMSCs), and the generated metabolic intermediate is, in a form of citrate, involved in bone formation, so that the 3D porous scaffold is beneficial to the functions of osteogenesis and angiogenesis in the bone repair process, shortens the bone defect repair time, and has a great application prospect in the field of bone tissue regeneration and repair, particularly in the aspect of large-sized bone defect repair.
5. The bioenergetic-active substance 3-hydroxybutyric acid (3HB) generated by the degradation of the bioenergetic-active material of the present invention is, in the form of citrate, involved in bone formation via a tricarboxylic acid metabolic cycle, and also can induce angiogenesis, and has an important application prospect in the field of preparation of vascularized bone regeneration materials, large-sized bone defect repair materials or critical bone defect repair materials.

### BRIEF DESCRIPTION OF THE DRAWINGS

FIG. 1 is a gel permeation chromatogram of poly(3-hydroxybutyrate-co-4-hydroxybutyrate), where A represents a standard curve, and B represents poly(3-hydroxybutyrate-co-4-hydroxybutyrate);

FIG. 2 is a diagram of the porous scaffold (A) manufactured by 3D printing and a cross-sectional scanning electron microscope image (B) thereof;

FIG. 3 shows the effect of the degradation product 3-hydroxybutyric acid of the scaffold on the proliferation of human bone mesenchymal stem cells (hBMSCs);

FIG. 4 shows the effect of the degradation product 3-hydroxybutyric acid of the scaffold on the expression activity of an alkaline phosphatase in hBMSCs;

FIG. 5 shows the effect of the degradation product 3-hydroxybutyric acid of the scaffold on the formation of extracellular calcium nodules in hBMSCs;

FIG. 6 shows the formation of extracellular apatite in hBMSCs by Confocal Raman Spectroscopy;

FIG. 7 shows the effect of the degradation product 3-hydroxybutyric acid of the scaffold on the expression of osteogenic differentiation-related genes in hBMSCs;

FIG. 8 shows the effect of the degradation product 3-hydroxybutyric acid of the scaffold on the mitochondrial membrane potential of cells;

FIG. 9 shows the formation of metabolic intermediates generated by ¹³C-labeled 3HB involved in TCA cycle through LC-MS/MS metabolic flux analysis;

FIG. 10 shows quantitative analysis of metabolic intermediates in TCA cycle by LC-MS/MS;

FIG. 11 shows the analysis of citrate content in culture supernatant;

FIG. 12 shows the changes of body weight and uterus in ovariectomy-induced osteoporosis;

FIG. 13 shows the analysis of 3HB on bone mass by micro-CT in ovariectomy-induced osteoporosis;

FIG. 14 shows silver nitrate staining (a) and analysis of bone formation by double fluorescence labeling (c-e);

FIG. 15 shows the analysis of the form of 3HB existing in bone in vivo (a-c) and the content of citrate (d) and calcium (e) in serum were determined;

FIG. 16 shows the establishment of a critical bone defect model of a rat skull;

FIG. 17 shows the analysis of the effect of 3HB on EA.hy926 cell migration by wound healing assay;

FIG. 18 shows the analysis of microtube formation of EA.hy926 induced by 3HB in vitro;

FIG. 19 shows the analysis of angiogenesis in the bone defect region (a, b) through high-resolution multiphoton microscopy; and

FIG. 20 shows the 12-week post-evaluation on the regeneration of the neogenetic bone tissue at the bone defect through micro-CT.

### DESCRIPTION OF THE EMBODIMENTS

In order to understand the present invention more clearly, the present invention will be further described with reference to the following examples and drawings. The examples are given for the purpose of illustration only and are not intended to limit the present invention in any way. In the examples, all of the raw reagent materials are commercially available, and the experimental method without specifying the specific conditions is a conventional method and a conventional condition well known in the art, or a condition suggested by an instrument manufacturer.

### Example 1

(1) Microorganism halophilic monads capable of synthesizing poly(3-hydroxybutyrate-co-4-hydroxybutyrate) were fermented and cultured in a 60 MMG culture medium at 37° C. and 400-800 rpm for 72 hours, and thalli were collected after 72 hours, and then ventilated and dried at 70° C. to obtain dry thalli powder containing poly(3-hydroxybutyrate-co-4-hydroxybutyrate).

The 60 MMG culture medium consists of 30 g/L of glucose, 1 g/L of yeast extract, 0.25 g/L of ammonium sulfate, 0.2 g/L of magnesium sulfate, 9.65 g/L of disodium hydrogen phosphate, 1.5 g/L of potassium dihydrogen phosphate, 10 mL/L of trace element I, and 1 mL/L of trace element II.

(2) poly(3-hydroxybutyrate-co-4-hydroxybutyrate) of the dry thalli powder was extracted with chloroform (20 mL of chloroform was added into 1 g of dry thalli powder), uniformly stirred, then placed into a high-pressure reaction kettle, and reacted at 100° C. for 4 hours.

(3) After the high-pressure reaction kettle was cooled, cell debris were removed by adopting a filtering or suction filtration method to obtain a clear chloroform solution.

(4) The chloroform solution was concentrated at 60° C. (at a rate of 100 mL of chloroform solution to 60 mL), and then added into 15 volumes of pre-cooled absolute ethanol, and the reaction mixture was placed in a refrigerator at 4° C. overnight for precipitation.

(5) The precipitate obtained in the step (4) was filtered and collected, the collected precipitate was placed in a vacuum drying oven at 40° C. for 24 hours, and after the solvent was completely volatilized, poly(3-hydroxybutyrate-co-4-hydroxybutyrate) was obtained.

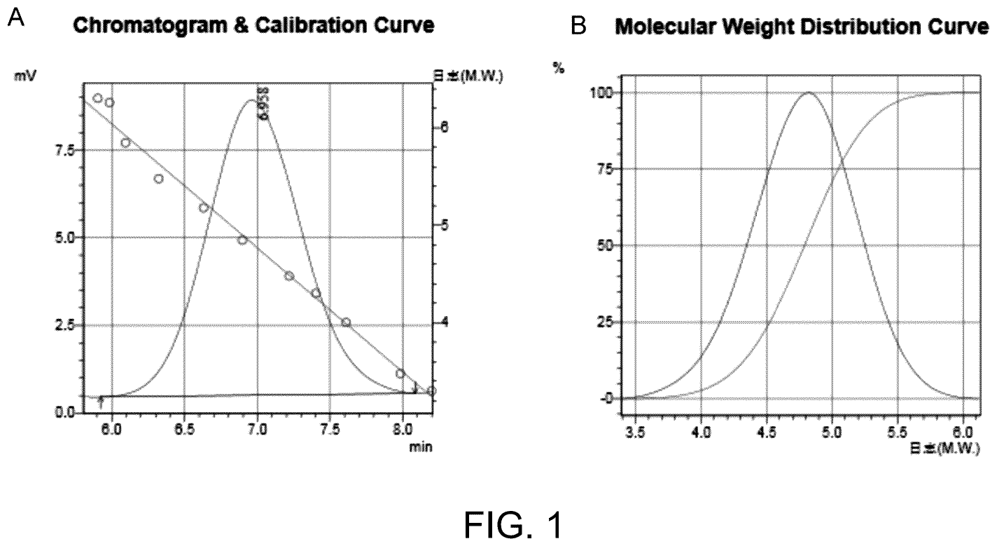

50 mg of poly(3-hydroxybutyrate-co-4-hydroxybutyrate) was weighed and dissolved in chloroform, and left to stand for 1 hour to form a uniform solution, and then 10 μL of the solution was taken and subjected to gel permeation chromatography to test the molecular weight. The results are shown in FIG. 1.

The calculation results of the molecular weight were as follows:

GPC Calculation Results
Peak #: 1 (DetectorA Ch1) [symbol]
[Peak information]

[symbol]

| | | | Molecular | |
|---|---|---|---|---|
| | Time(min) | Volume(mL) | Weight | Height |
| Start | 5.925 | 5.925 | 1362905 | 459 |
| Top | 6.958 | 6.958 | 65994 | 8418 |
| End | 8.083 | 8.083 | 2435 | 565 |

Area: 383862
Area %: 100.0000

[Average molecular weight]

[symbol]

| | |
|---|---|
| Number Average Molecular | 40265 |
| Weight(Mn) | |
| Weight Average Molecular | 89532 |
| Weight(Mw) | |
| Z Average Molecular | 179321 |
| Weight(Mz) | |
| Z + 1 Average Molecular | 312585 |
| Weight(Mz1) | |
| Mw/Mn | 2.22354 |
| Mv/Mn | 0.00000 |
| Mz/Mw | 2.00287 |

Detector A Ch1 [symbol]
[Average molecular weight (total)]

[symbol]

| | |
|---|---|
| Number Average Molecular | 40265 |
| Weight(Mn) | |
| Weight Average Molecular | 89532 |
| Weight(Mw) | |
| Z Average Molecular | 179321 |
| Weight(Mz) | |
| Z + 1 Average Molecular | 312585 |
| Weight(Mz1) | |
| Mw/Mn | 2.22354 |
| Mv/Mn | 0.00000 |
| Mz/Mw | 2.00287 |

[symbol]

indicates data missing or illegible when filed

### Example 2

The poly(3-hydroxybutyrate-co-4-hydroxybutyrate) material synthesized in Example 1 was put into a fused 3D) printer (180° C.) to manufacture a porous scaffold for bone tissue repair.

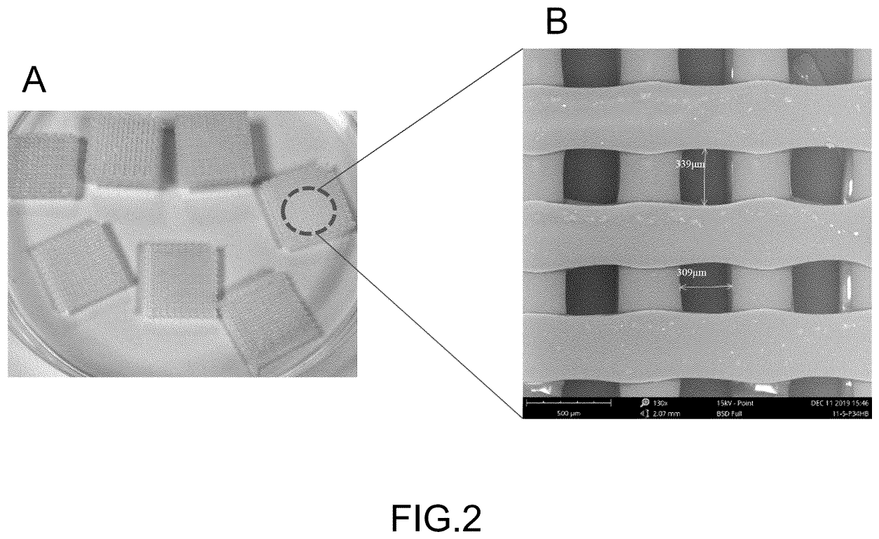

The results are shown in FIG. 2, where A of FIG. 2 is a diagram of the porous scaffold manufactured by 3D) printing, and B of FIG. 2 is a cross-sectional scanning electron microscope image of the scaffold, with a pore diameter of the porous scaffold being about 350-400 μm.

### Experimental Example 1

The 3D porous scaffold of Example 2 was soaked in a phosphate buffer for 8 weeks and the degradation products were collected. In the phosphate buffer, the main degradation product of poly(3-hydroxybutyrate-co-4-hydroxybutyrate) is 3-hydroxybutyric acid (3HB). The concentration of the collected degradation products was determined, and then an in-vitro experiment was performed.

The phosphate buffer consists of 7.9 g/L of sodium chloride, 0.2 g/L of potassium chloride and 0.24 g/L of monopotassium phosphate.

(1) Human bone mesenchymal stem cells (hBMSCs) in a good growth state were seeded onto a 48-well plate at a cell density of 2×10⁴, 3-hydroxybutyric acid at different concentrations (0 μM, 10 μM, 40 μM, 80 μM, 160 μM, 320 μM) was added into the cells after 4 hours, and cell proliferation was determined on days 1, 5, and 7 with CCK-8, respectively. Lactic acid (LA) was also taken as a control.

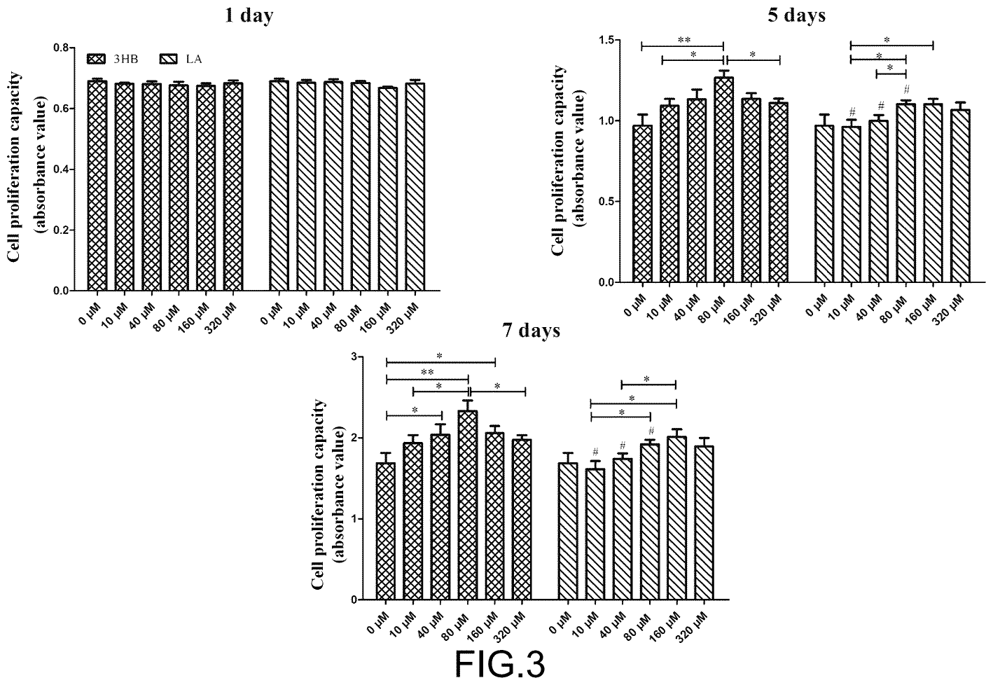

The results are shown in FIG. 3. FIG. 3 shows the effect of the degradation product 3-hydroxybutyric acid of the scaffold on the proliferation of human bone mesenchymal stem cells (hBMSCs); compared with the control group (LA), the proliferation of the bone mesenchymal stem cells was significantly enhanced after 3HB stimulation on days 5 and 7, which indicates that the degradation products of the scaffold can promote the proliferation of the hBMSCs.

(2) Human bone mesenchymal stem cells (hBMSCs) were seeded onto a 6-well plate at a cell density of 1×10⁵ and cultured for 12 hours. Osteoinductive differentiation solutions containing 3-hydroxybutyric acid at different concentrations (0 μM, 40 μM, 160 μM, 320 μM) were added to the cells, and the expression of an alkaline phosphatase was detected on days 7 and 14, respectively. The expression of the alkaline phosphatase was detected according to the instructions of an activity detection kit for the alkaline phosphatase.

The osteoinductive differentiation solution consists of low-sugar DMEM medium+10% fetal bovine serum+2 mM L-glutamine+100 U/mL penicillin+100 μg/mL streptomycin+100 nM dexamethasone+0.2 mM L-ascorbic acid+10 mM β-sodium glycerophosphate.

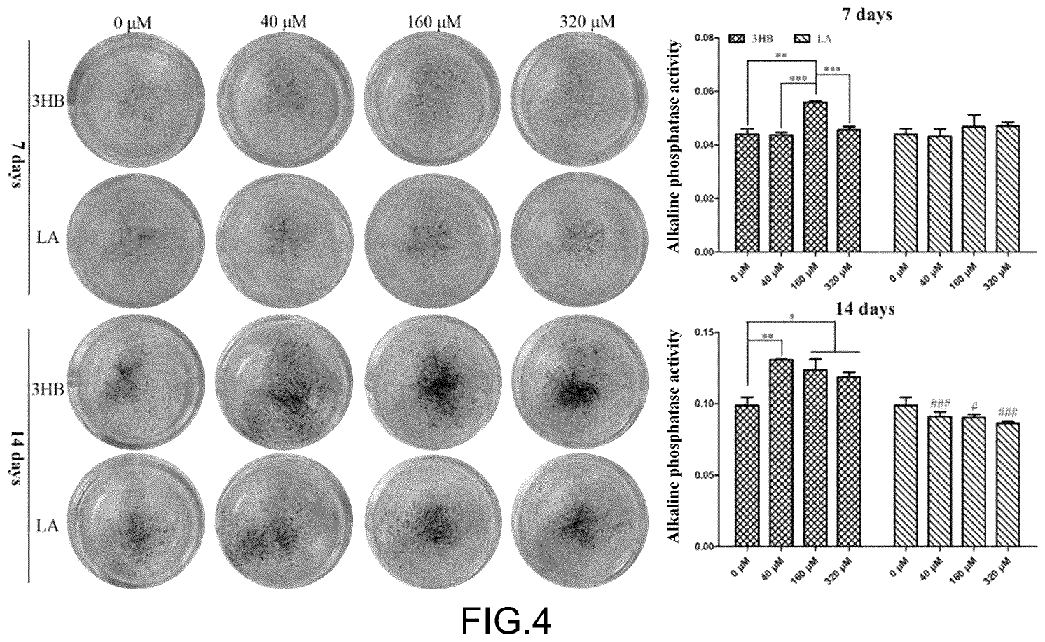

The results are shown in FIG. 4. FIG. 4 shows the effect of the degradation product 3-hydroxybutyric acid of the scaffold on the expression activity of an alkaline phosphatase in the hBMSCs, which indicates that the degradation product of the scaffold can promote the expression of the alkaline phosphatase in the hBMSCs. Compared with the control group (LA), the expression of the alkaline phosphatase was significantly enhanced after the bone mesenchymal stem cells were induced to differentiate for 14 days under the stimulation of the 3HB, while the control group showed an inhibitory effect.

(3) Human bone mesenchymal stem cells (hBMSCs) were seeded onto a 6-well plate at a cell density of 1×10⁵ and cultured for 12 hours. Osteoinductive differentiation solutions containing 3-hydroxybutyric acid at different concentrations (0 μM, 40 μM, 160 μM, 320 μM) were added to the cells, and the formation of extracellular calcium nodules was detected by using alizarin red on day 10 and 14 respectively.

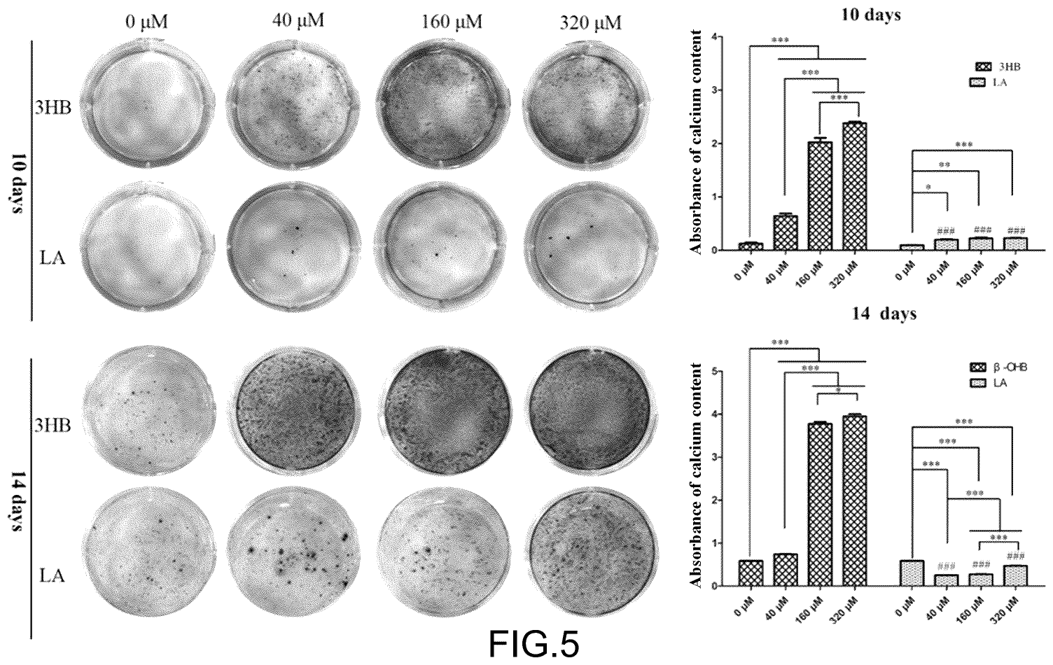

The results are shown in FIG. 5. FIG. 5 shows the effect of the degradation product 3-hydroxybutyric acid of the scaffold on the formation of extracellular calcium nodules in hBMSCs; compared with the control group (LA), the deposition of extracellular calcium nodules was significantly enhanced after the bone mesenchymal stem cells were induced to differentiate for 10 days and 14 days under the stimulation of the 3HB respectively, which indicates that 3-hydroxybutyric acid promotes the formation of calcium nodules of the bone mesenchymal stem cells as osteogenic differentiation markers.

(4) Human bone mesenchymal stem cells (hBMSCs) were seeded onto a 6-well plate at a cell density of 1×10⁵ and cultured for 12 hours. Osteoinductive differentiation solutions containing 3-hydroxybutyric acid at different concentrations (0 μM, 40 μM, 160 μM, 320 μM) were added to the cells and the cells were induced to differentiate for 21 days, and after 21 days of induction, the formation of extracellular apatite was detected by Confocal Raman Spectroscopy.

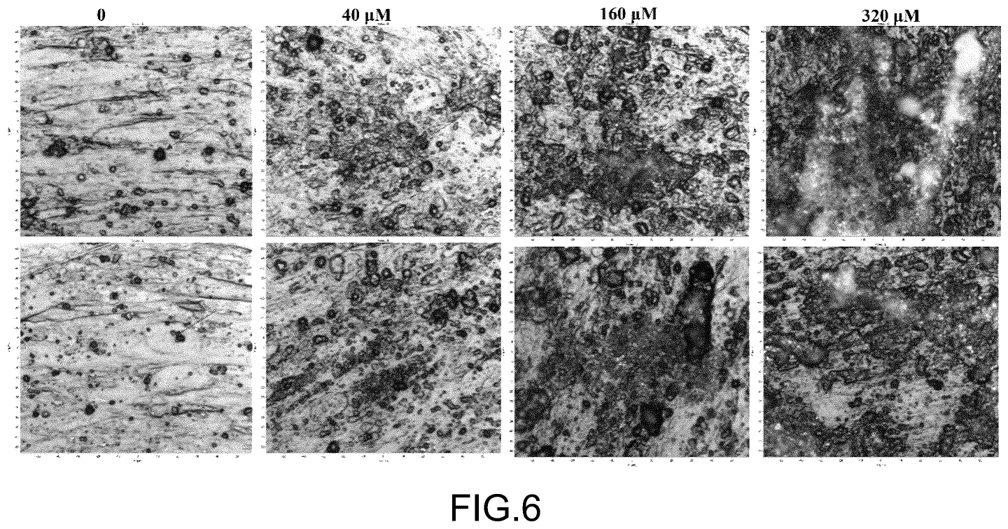

The results are shown in FIG. 6. FIG. 6 shows the formation of extracellular apatite in hBMSCs by Confocal Raman Spectroscopy, where after 21 days of induced differentiation of the bone mesenchymal stem cells, a large amount of extracellular apatite was formed and the content of apatite increased with the increase of the 3HB concentration, which indicates that 3-hydroxybutyric acid can promote the formation of osteogenic differentiation apatite in the bone mesenchymal stem cells.

(5) Human bone mesenchymal stem cells (hBMSCs) were seeded onto a 6-well plate at a cell density of 1×10⁵ and cultured for 12 hours. Osteoinductive differentiation solutions containing 3-hydroxybutyric acid at different concentrations (0 μM, 40 μM, 160 μM, 320 μM) were added to the cells and the cells were induced to differentiate for 7 days, and after 7 days of induction, the expression of osteogenic differentiation-related genes (Runx-related transcription factor 2 gene, osteocalcin gene and osteoprotegerin gene) was detected by utilizing a real-time quantitative fluorescent PCR technology.

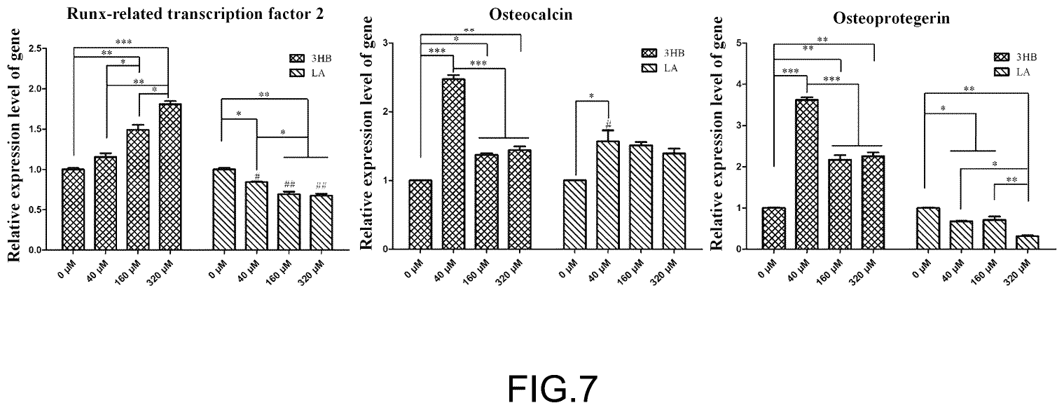

The results are shown in FIG. 7. FIG. 7 shows the effect of the degradation product 3-hydroxybutyric acid of the scaffold on the expression of osteogenic differentiation-related genes in hBMSCs; compared with the control group (LA), the expression of osteogenic differentiation-related genes was enhanced after the bone mesenchymal stem cells were induced to differentiate for 7 days under the stimulation of the 3HB, while the control group inhibited the expression of the genes to a certain extent, which indicates that the degradation product 3-hydroxybutyric acid of the scaffold can promote the expression of the osteogenic differentiation-related genes in hBMSCs.

(6) Human bone mesenchymal stem cells (hBMSCs) were seeded onto a 6-well plate at a cell density of 1×10⁴ and cultured for 12 hours. There were three groups, including a positive control group, a negative control group and an experiment group, wherein the cells of the positive control group were treated with high-sugar culture medium (HG) for 6 hours; the cells of the negative control group were treated with high-sugar culture medium containing an oxidative phosphorylation uncoupler (CCCP) for 6 hours; the cells of the experimental group were treated with a sugar-free culture medium (GF) containing 3-hydroxybutyric acid at different concentrations (40 μM, 160 μM, 320 μM) and lactic acid (LA) for 6 hours.

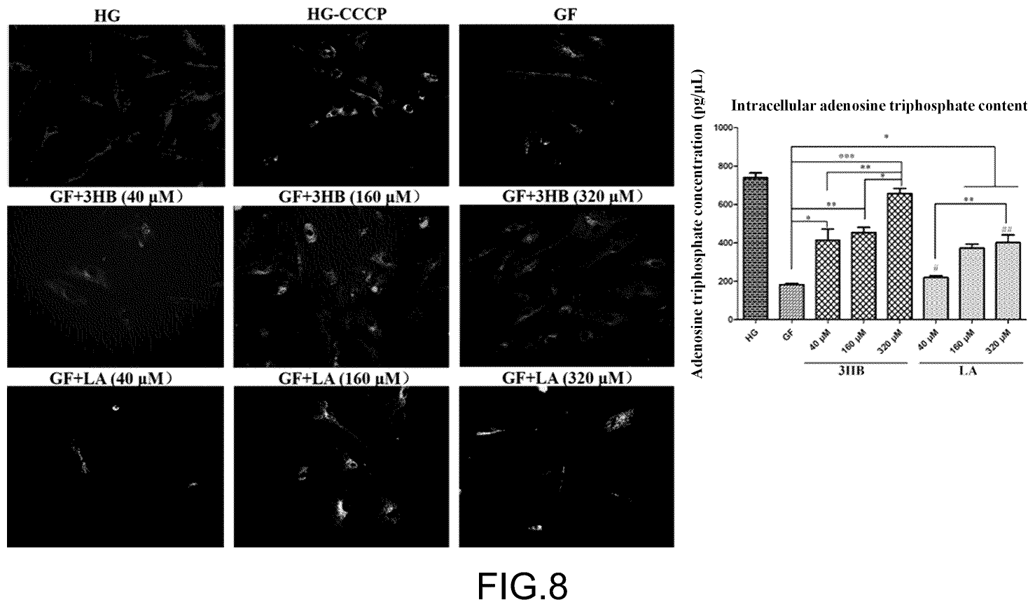

The results are shown in FIG. 8. FIG. 8 shows the effect of the degradation product 3-hydroxybutyric acid of the scaffold on the mitochondrial membrane potential of cells, where the results show that the degradation product 3-hydroxybutyric acid of the scaffold can provide ATP for the cells and maintain the mitochondrial membrane potential (ΔΨm), and compared with the LA group, the 3HB can provide more bioenergy (ATP) for the cells and increase the cell membrane potential.

### Experimental Example 2

In this experimental example, the metabolic flux of the 3HB and the manner of being involved in bone formation were analyzed in a ¹³C-labeled 3HB tracer experiment, where the cells not treated with ¹³C-labeled 3HB were taken as a control group. The human bone mesenchymal stem cells (hBMSCs) were seeded onto a culture plate, and an osteoinductive differentiation solution containing 1 mM ¹³C-3HB was added into the cells, followed by metabolomics analysis (LC-MS/MS) on day 14.

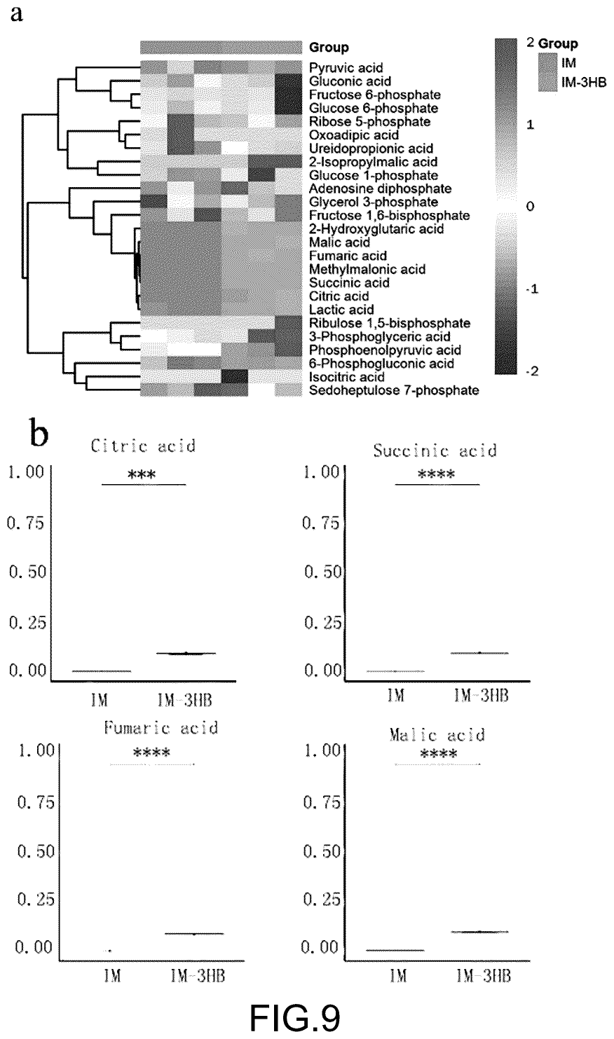

After the hBMSCs were induced for osteogenic differentiation for 14 days under the stimulation of 1 mM ¹³C-3HB, metabolomics analysis (LC-MS/MS) found that a number of metabolic intermediates (citrate, succinate, fumarate and malate) containing carbon atoms derived from ¹³C-labeled 3HB were detected in the TCA cycle, whereas no carbon atom derived from ¹³C-labeled 3HB was found in the control group (not treated with ¹³C-labeled 3HB) (FIG. 9), which demonstrates that the bioenergetic-active substance 3HB produced by degradation of the P34HB scaffold can be involved in the metabolism via the TCA cycle.

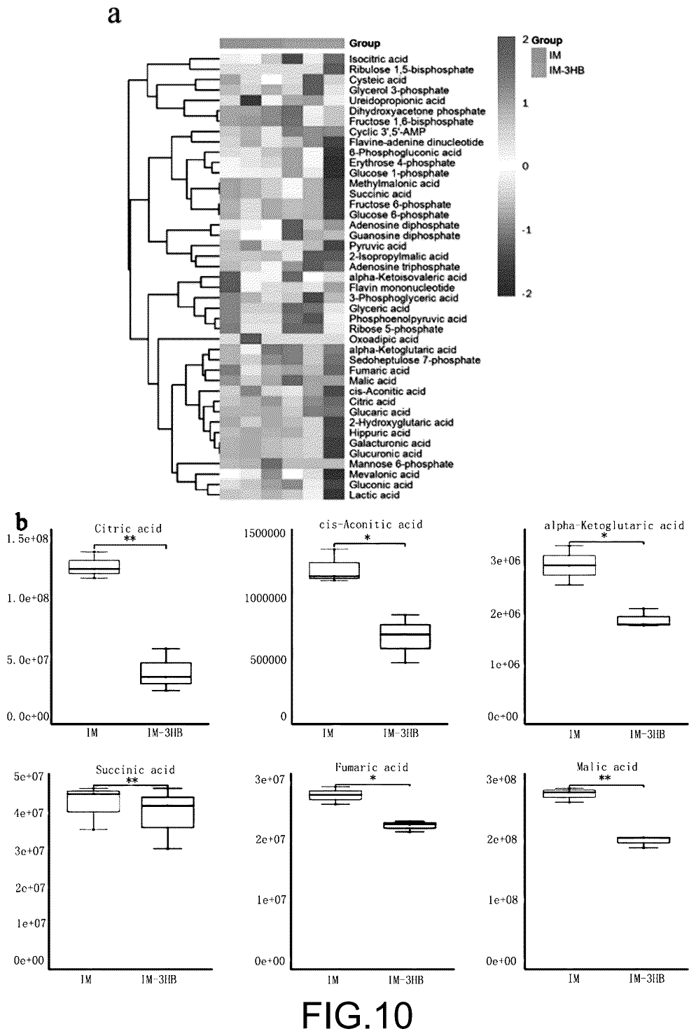

Furthermore, through relative quantitation of the total content of metabolites in the intracellular TCA cycle, the results showed that compared with the control group, after treatment with 1 mM ¹³C-labeled 3HB, the contents of various intracellular metabolic intermediates such as citrate, isocitrate, fumarate and malate were significantly lower than that of the untreated group (FIG. 10). This result seemed to be contradictory to the effect of ¹³C-labeled 3HB being involved in the TCA metabolic cycle to increase the content of the metabolic intermediates, and also contradictory to the effect of the 3HB capable of up-regulating the oxidative phosphorylation function, promoting the osteogenic differentiation of the hBMSCs, and promoting the formation of extracellular calcium deposition.

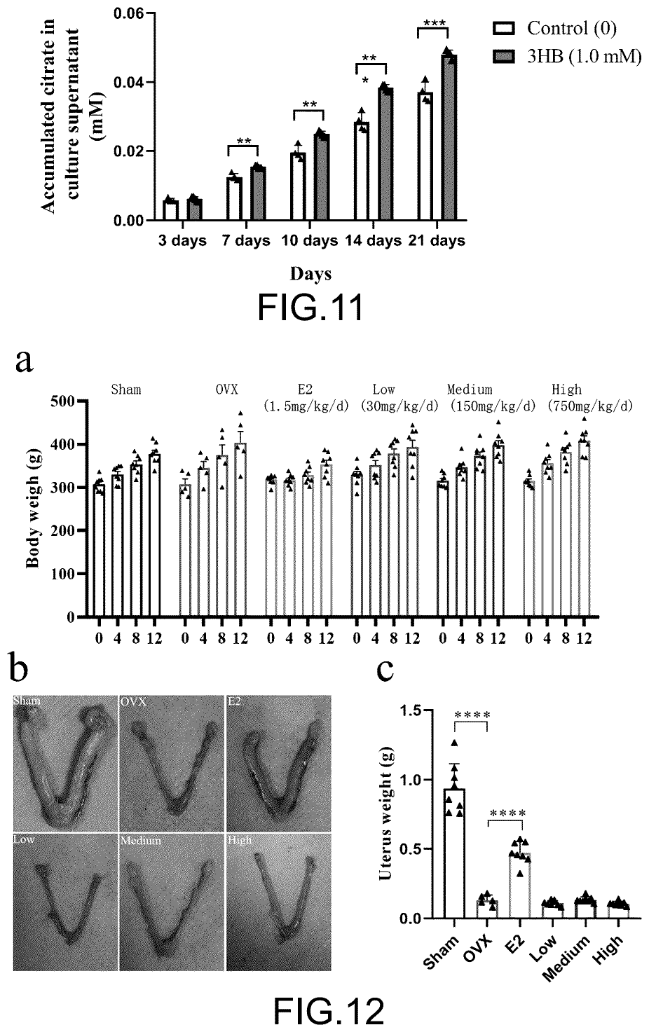

Furthermore, the osteoinductive differentiation cell supernatant was analyzed. It was found that more citrate was detected in the supernatant in the ¹³C-labeled 3HB treatment group than in the control group (FIG. 11). This result explained that the bioenergetic-active substance 3HB did not increase the content of the metabolic intermediates in cells although being involved in the TCA cycle. The reason is that after the treatment with ¹³C-labeled 3HB, citrate formed via TCA was transferred from the inside of the cells to the outside of the cells to be involved in calcium matrix deposition, thereby causing a decrease in the intracellular citrate content. However, citrate is a key intermediate in the TCA cycle, and has to be converted, in order to maintain the basic metabolic activity of cells, from other substances in the TCA cycle such as succinate and fumarate, thereby reducing the accumulation of metabolic intermediates throughout the TCA cycle.

The above results show that the bioenergetic-active substance 3HB is involved, in the form of a metabolic intermediate citrate, in the in-vitro biomineralization formation via the TCA cycle.

### Experimental Example 3

In this experimental example, the mechanism of promoting bone regeneration by the 3HB in vivo is elucidated by taking the 3HB as a medium and taking an osteoporosis model of an ovariectomized rat as a research object.

The results showed that the weight gain of the ovariectomized rat was faster than that of the sham group, while the weight gain in the E2 group was comparable to that of the sham group (a of FIG. 12), which was due to the increase in adipose tissue caused by the fact that the imbalance of estrogen in the rat body after ovariectomy and thus the rat could not exert a potent regulatory function. After 3 months of continuous lavage administration, the uteruses of each group of experimental rats were sampled. It was found that the uteruses of the rats in the OVX group and the 3HB gavage group exhibited a distinct atrophy state, the uterus atrophy of the E2 group was improved, and the uteruses of the sham group exhibited a normal morphology (b of FIG. 12). In addition, the uterine weights of the rats in the OVX group and the 3HB gavage group were also significantly lower than those of the sham group and the E2 group (c of FIG. 12). The experimental data show that the osteoporosis model of the ovariectomized rat is successfully established, and a foundation is laid for the subsequent research of 3HB regulation and control of bone regeneration.

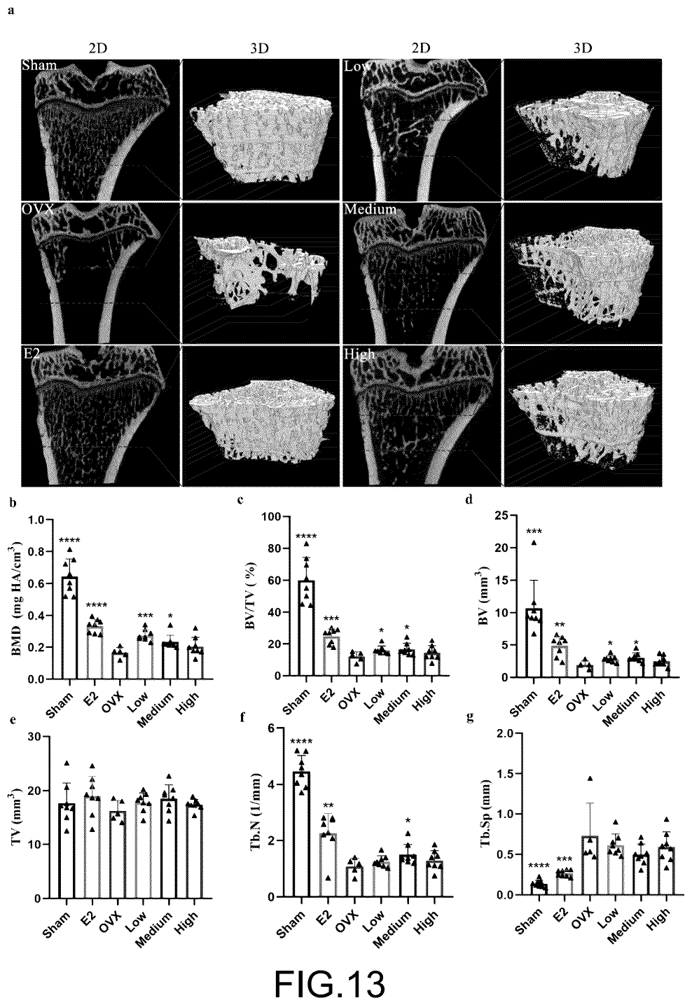

Micro-CT scans of small animals revealed that different doses of 3HB exhibited some slowing of bone loss in ovariectomized rats after lavage administration of 3HB for 3 consecutive months (a of FIG. 13). Compared with the OVX group, when ovariectomized rats were administrated low-dose (30 mg/kg/d) and medium-dose (150 mg/kg/d) of 3HB, bone density (BMD), bone volume fraction (BV/TV) and bone volume (BV) significantly increased (b to d of FIG. 13). However, the number of trabecular bones (Tb.N) showed the highest in the medium-dose 3HB gavage group, which was significantly more than in the OVX group (f of FIG. 13). Although the tissue volume (TV) showed no statistical difference between groups, it also increased to varying degrees compared to the OVX group (e of FIG. 13). In addition, the spacing between trabecular bones (Tb.Sp) also decreased to varying degrees after administration of different doses of 3HB (g of FIG. 13).

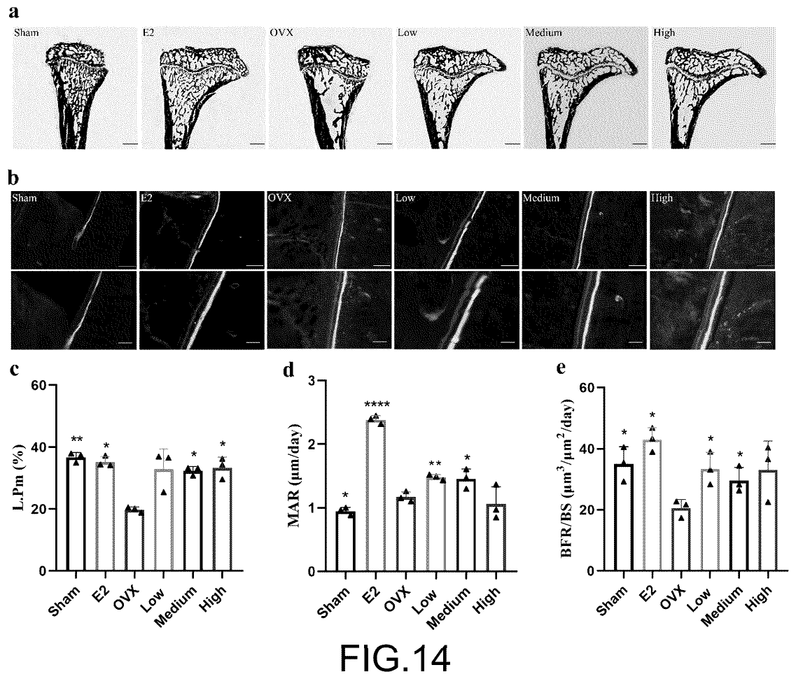

Silver nitrate staining of tibial tissue sections revealed that the number of trabecular bones was greater than that in the OVX group after administration of different doses of 3HB, while the number of trabecular bones showed no significant difference between the 3HB groups with different doses (a of FIG. 14), whereas the number of trabecular bones largely increased after treatment with E2, but the number thereof was still lower than that in the sham group, as also seen from the results of micro-CT quantitative analysis (f of FIG. 14). Calcein/xylenol orange dual fluorescent labeling was then performed in vivo to analyze bone tissue regeneration rate and mineral characteristics (b of FIG. 14). Research shows that the percent fluorescence perimeter of calcein/xylenol orange bifluorescent markers in the 3HB groups with different doses was higher than that in the OVX group, and the percent fluorescence perimeter of calcein/xylenol orange bifluorescent markers in the medium-dose and high-dose 3HB groups had statistical difference compared to the OVX group. However, there was no difference in the percent fluorescence perimeter of calcein/xylenol orange bifluorescent markers between the 3HB groups with different doses, the sham group and the E2 group (c of FIG. 14). In addition, similar results were shown in mineral apposition rate (MAR) and bone formation rate (BFR/BS). Compared with the OVX group, the low-dose and medium-dose 3HB treatment groups showed significant MAR and BFR/BS (d of FIG. 14, e of FIG. 14). However, the E2 treatment group showed the highest MAR and BFR/BS, and the sham group showed the MAR and BFR/BS at a normal level.

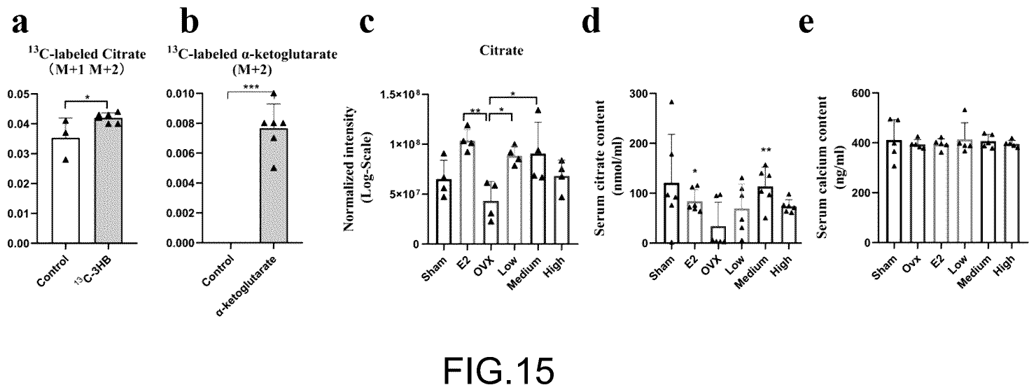

The results of the ¹³C-labeled 3HB tracer experiment showed that there were ¹³C-labeled citrate and α-ketoglutarate in bone tissues in the low-dose ¹³C-labeled 3HB gavage group as detected by LC-MS/MS (a of FIG. 15, b of FIG. 15). Meanwhile, the content of citrate in bone tissues was measured, and it was found that 3HB promoted the formation of citrate in bone tissues (c of FIG. 15). In addition, serological tests found that the bioenergetic-active substance 3HB can increase the content of citrate in serum (d of FIG. 15). However, no significant change in the content of calcium in serum was found (e of FIG. 15). Therefore, the decrease in bone mass in the ovariectomized rats may not be caused by the decrease in the content of calcium in serum, but may be caused by the fact that citrate may not effectively bind to calcium and phosphorus due to the decrease in the content of citrate in bone.

The above experimental results show that the bioenergetic-active substance 3HB generated by degradation of the P34HB can improve the in-vivo mineral apposition rate and the bone formation rate and reduce the loss of bone mass of the osteoporosis rats. It is involved, in the form of citrate, in the bone formation and can improve the osteoporosis symptom.

### Experimental Example 4

The above experimental example shows that the bioenergetic-active substance 3HB generated by degradation of the P34HB can promote the proliferation of the hBMSCs, mediate the oxidative phosphorylation of mitochondria to promote osteogenic differentiation, and be involved, in the form of a metabolic intermediate citrate in the TCA cycle, in bone regeneration. Therefore, the P34HB is an excellent candidate material for bone regeneration, and shows great application potential in the field of bone tissue engineering regeneration. The repair of bone injury is a pathological and physiological process of proliferation, migration and differentiation of various cells such as stem/progenitor cells and vascular endothelial cells driven by bioenergy, which allows bone regeneration and angiogenesis to proceed in an orderly manner through a complex signal regulation network. In order to achieve the regeneration and functional reconstruction of the large-sized bone defect as quickly as possible, a complete vascular network has to be established as early as possible between the graft and the surrounding tissue to provide the oxygen and nutrients required for bone regeneration. In this experimental example, the role and function of the P34HB bioenergy scaffold in promoting the vascularized bone regeneration were researched by taking a 3D-printed P34HB bioenergy scaffold as a medium, taking human umbilical vein fusion cells (EA.hy926) as a cell model in vitro and taking critical bone defect of a rat skull as an animal model in vivo.

### I. Experimental Method and Procedure

### (I) Effect of Bioenergetic-Active Substance 3HB Generated by Degradation of P34HB on EA.Hy926 Cell Migration

(1) The EA.hy926 cells were seeded onto a 6-well plate at a density of 5×10⁴/mL, and cultured in a cell culture box;
(2) After the cell growth confluence was close to 100%, the culture medium was removed and rinsed 3 times with PBS;
(3) Cell scratches were made along the centers of plate wells by using a 200 μL pipette tip, the plate was rinsed 3 times with PBS to remove the scratched cells;
(4) The cells were treated with sugar-free culture media containing different concentrations of 3HB (with 1% FBS) for 10 hours and 20 hours, respectively;
(5) The migration of the cells was observed by using a microscope at a set time point and then photographed; and
(6) The quantitative analysis was performed on cell migration by using ImageJ software.

### (II) Effect of Bioenergetic-Active Substance 3HB Generated by Degradation of P34HB on Microvessel Formation of EA.Hy926 Cells

(1) Matrigel matrix was thawed in a refrigerator at 4° C. overnight in advance, and a pre-cooled pipette tip and an angiogenesis chamber were prepared;
(2) The angiogenesis chamber was added with the thawed Matrigel matrix, and placed into a cell culture box for 30 minutes at a volume of 10 μL per well;
(3) A cell suspension of EA.hy926 was prepared at a density of 3×10⁵/mL, and added into the angiogenesis chamber at a volume of 50 μL per well;
(4) Within a set time point (6 h), the formed microvascular network was stained with calcein and then photographed; and
(5) The quantitative analysis was performed on the formed microvascular network by using ImageJ software.

### (III) Establishment of Critical Bone Defect Model of Rat Skull

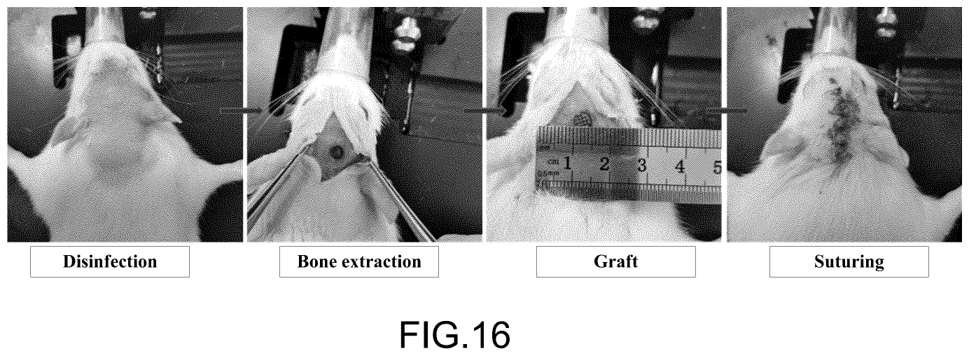

Male SD rats (10 weeks old) purchased from Beijing Vital River Laboratory Animal Technology Co., Ltd. were used in this experiment. All experimental rats were bred in the animal center (SPF) of Shenzhen Institute of Advanced Technology, Chinese Academy of Sciences, and the animal experiments of the paper were approved by the Ethical Review Board of Shenzhen Institute of Advanced Technology, Chinese Academy of Sciences (SIAT-IACUC-201010-KYC-ZP-A1416). The process (FIG. 16) for establishing a critical bone defect model of SD rat skull comprises the following steps: all rats were anesthetized with 3.5% isoflurane-100% oxygen during each surgery, and simultaneously, and the heat preservation and dehydration prevention treatment were performed, including adding distilled water into a breathing anesthesia pipeline and injecting normal saline (500 μL) before the surgery. After anesthesia of animals, the skin of the surgical site was disinfected with iodine tincture and 75% alcohol, an incision of about 10 mm was made in the parietal skin along the sagittal suture to expose the left parietal bone, the periosteum was pushed away with sterile cotton swabs, the skull with a diameter of 5 mm was removed by using a high-speed dentist drill equipped with a circular saw under heat dissipation of physiological saline, and the endocrania could not be damaged during the removal of bone pieces. After different experimental treatments, the skull membrane and the head skin were placed to their right places, and the skin was sutured.

### (IV) Animal Grouping and Scaffold Graft

The critical bone defect models of rat skulls were randomly divided into three groups, 6 rats per group, group A: no treatment was done at the bone defect site (Empty); group B: PLLA scaffold graft group (PLLA); group C: P34HB scaffold graft group (P34HB). After scaffold implantation, the rats were administrated normal feed and water.

### (V) Angiogenesis at Bone Defect Sites Through High-Resolution Multiphoton Microscopy

12 weeks after surgery, the dynamic process of angiogenesis and development during the repair of bone defects, including changes in the number of angiogenesis, blood vessel diameter, blood vessel density, and blood vessel morphology, was monitored and quantified in vivo by using the high-resolution multiphoton microscopy established by the research group earlier. In order to further improve the contrast of the fluorescence signal of the blood vessels in the skull, FITC-Dextran (300 μL, 2 mg/mL) was injected into the tail vein of the rats. The pixel of each imaged picture was 256×256, the size of the picture is 512 μm×512 μm, and the time for acquiring each image is 8 seconds. Firstly, a clear imaging part was found by aiming at a focal length, X-Y plane imaging was carried out firstly, then the focal length was properly adjusted, and further scanning imaging was carried out along the Z-axis direction, with the scanning depth being 200 μm. The angiogenesis coupling ability of the P34HB scaffold during bone regeneration was evaluated.

### (VI) Micro-CT Analysis of Effect of P34HB Scaffold on Bone Regeneration at Defect Sites

12 weeks after surgery, the bone regeneration at the bone defect sites, including bone density (BMD), BV/TV, Tb.N, Tb.Th and other bone-related indexes, was analyzed by using micro-CT to evaluate the efficiency of the P34HB scaffold in repairing the bone defect.

### (VII) Statistical Analysis

Statistical analysis of the measured data was performed using GraphPad Prism 8.0 software. Data were analyzed using one-way ANOVA or T-test. The results were expressed as mean±standard deviation (SD). * P<0.05 represents a statistical difference.

### II. Results and Discussion

(I) Promotion of Bioenergetic-Active Substance 3HB Generated by Degradation of P34HB to Migration of EA.hy926 Cells

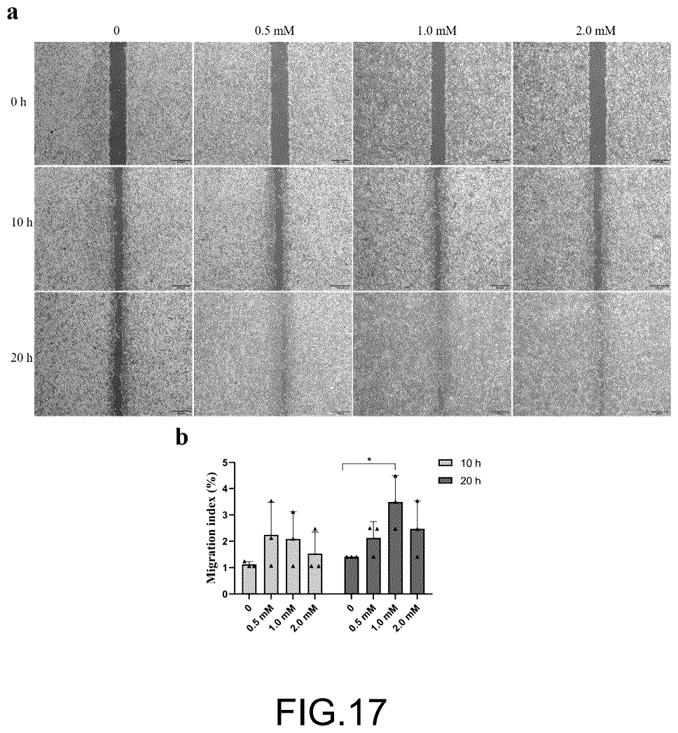

Bone regeneration is a complex physiological process involving a variety of cells, and cell migration plays an important role in regulating and controlling tissue regeneration. In the process of bone regeneration, the migration of vascular endothelial cells is beneficial to promoting the regeneration and functional reconstruction of bone defect tissues. The wound healing assay showed that compared with the control group, the 3HB with different concentrations exhibited the promotion effect of cell migration to a certain extent after treating EA.hy926 cells for 10 hours. The migration-promoting capacity was further improved after EA.hy926 cells were treated with the 3HB for 20 hours, wherein 1.0 mM 3HB showed a significant migration-promoting capacity to EA.hy926 cells (FIG. 17). The above experiments show that the bioenergetic-active substance 3HB generated by degradation of the P34HB can promote the cell migration in the process of bone regeneration and be involved in the regeneration and repair of tissues.

(II) Promotion of Bioenergetic-Active Substance 3HB Generated by Degradation of P34HB to Microtube Formation of EA.y926

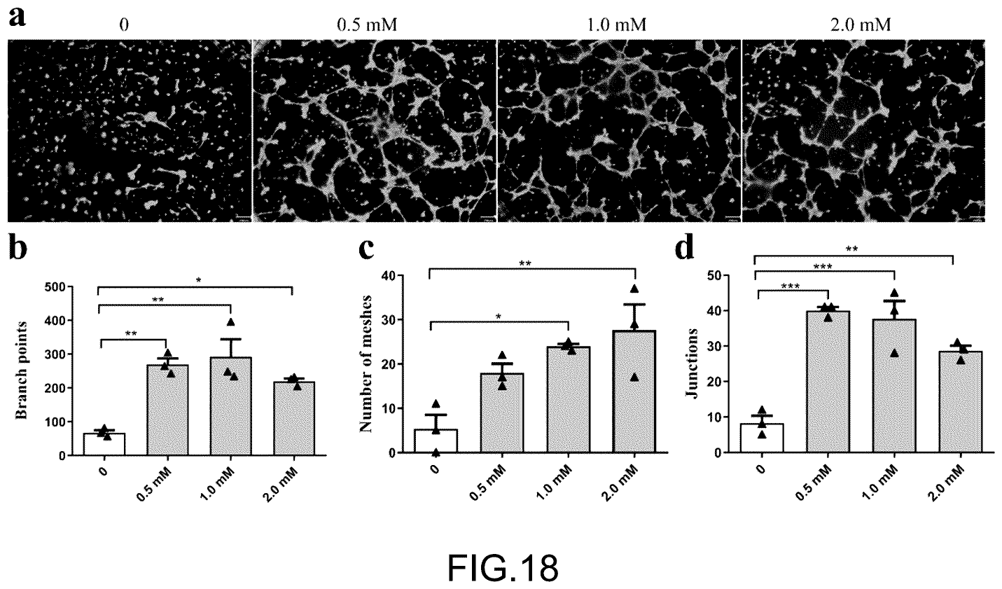

Bone tissue regeneration is a complex process based on the interaction between osteogenesis and angiogenesis. Angiogenesis is an essential part of the processes of bone formation, skeletal development and osseointegration, and is a prerequisite for cell survival and function. Since the normal vascular network function of the defect site is damaged, the necessary growth factors and nutrient substances cannot be provided for the tissue regeneration, thereby hindering the tissue regeneration and the functional reconstruction. The biological scaffold with the vascularization function is beneficial to the regeneration and functional reconstruction of bone defect tissues. The process of angiogenesis and development is also energy-consuming. The production of bioenergy (ATP) facilitates the vascularization of the biological scaffold. The in-vitro microvascular formation experiment showed that compared with the control group, more microvessel networks were formed after 6 hours of treatment of EA.hy926 cells with the bioenergetic-active substance 3HB generated by degradation of the P34HB (a of FIG. 18). Further quantitative analysis by ImageJ revealed that EA.hy926 cells treated with the bioenergetic-active substance 3HB significantly increased the number of microvascular branch points and the extent of microvascular junctions (b of FIG. 18, d of FIG. 18). Although the number of microvascular meshes exhibited no significant difference after 0.5 mM 3HB treatment, it increased to some extent (c of FIG. 18). The experimental results show that the bioenergetic-active substance 3HB generated by degradation of the P34HB can promote the migration of vascular endothelial cells and the formation of microvascular networks, which preliminarily indicates that the P34HB scaffold material has the vascularization potential.

### (III) Promotion of P34HB Bioenergy Scaffold to Vascularized Bone Formation for Repair of Critical Bone Defect of Rat Skull

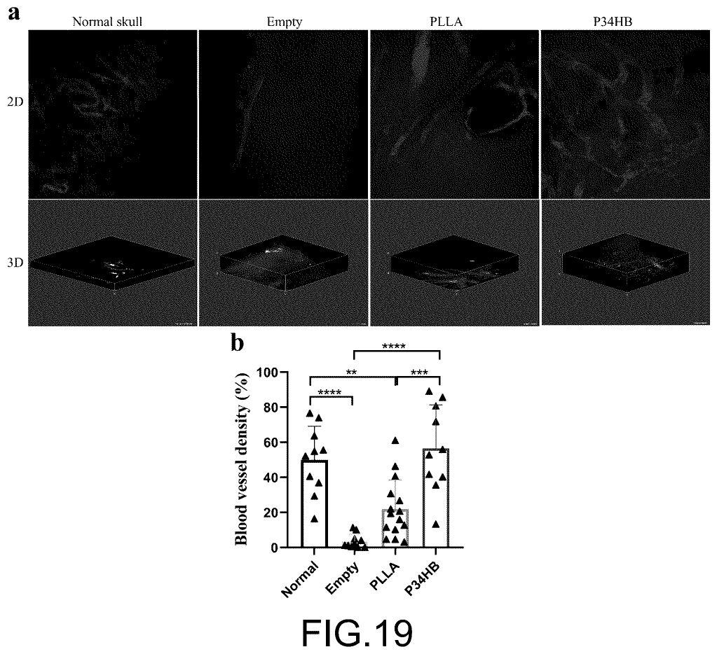

An ideal bone repair material needs not only to have osteoinductive regenerative capacity but also to meet the requirement of being able to vascularize early. Bone graft materials that do not have the vascularization capacity can result in necrosis of the graft due to ischemia. The above research shows that the P34HB bioenergy scaffold has a vascularization function. In order to explore the vascularization potential of the P34HB bioenergy scaffold, the angiogenesis coupling in the process of bone regeneration was observed by using a high-resolution multiphoton microscopy and taking a critical bone defect of a rat skull as a model. 12 weeks after surgery, as could be seen from the high-resolution multiphoton microscopy, the scaffold graft group showed more angiogenesis. However, the blood vessel density of the P34HB scaffold graft group was significantly higher than that of the PLLA scaffold graft group; the Empty group had almost no angiogenesis (a of FIG. 19). ImageJ quantitative analysis further supported the observed phenomenon described above (b of FIG. 19), which indicates that the P34HB is able to promote vascular regeneration in the process of bone regeneration.

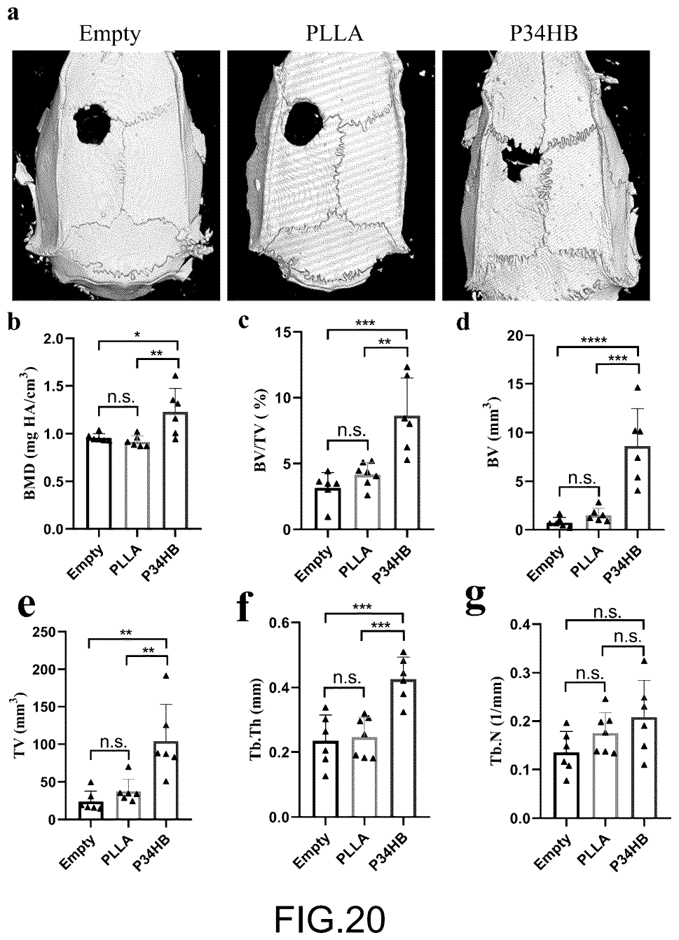

As can be seen from the high-resolution multiphoton microscopy, the P34HB bioenergy scaffold can promote the angiogenesis at bone defect sites. Subsequently, the bone regeneration after the scaffold graft was analyzed through the micro-CT of small animals. As can be seen from the Micro-CT, the bone defect site of the rat skull with a P34HB scaffold graft was filled with a large amount of new bone tissue, while the bone defect sites of the Empty group and the PLLA scaffold graft group were observed to have only a small amount of new bone tissue. According to the Micro-CT quantitative analysis, BMD, BV/TV, TV, BV and Tb.Th values of bone defect sites of the P34HB scaffold graft group were significantly higher than those of the Empty group and the PLLA scaffold graft group, and no significant difference existed between the Empty group and the PLLA scaffold graft group. However, no significant difference existed between the Empty group, the PLLA scaffold graft group and the P34HB scaffold graft group in terms of Tb.N. (FIG. 20)

The above experimental results show that the P34HB bioenergy scaffold can be coupled with angiogenesis in the process of bone regeneration, promote vascularized bone formation for repair of the critical bone defects, and achieve functional reconstruction.

1. A bioenergetic-active material, wherein the bioenergetic-active material is a biodegradable polymer; a degradation product of the bioenergetic-active material is a metabolic intermediate via a tricarboxylic acid cycle and/or a glycolysis pathway;
or a degradation product of the bioenergetic-active material is a polymeric monomer capable of being converted into a metabolic intermediate via a tricarboxylic acid cycle and/or a glycolysis pathway;
or a degradation product of the bioenergetic-active material is a polymer monomer capable of being converted into acetyl-coenzyme A.

2. The bioenergetic-active material according to claim 1, wherein the metabolic intermediate of the tricarboxylic acid cycle comprises one or more of citrate, aconitase, isocitrate, oxalosuccinate, α-ketoglutarate, succinyl-coenzyme A, succinate, fumarate, malate, and adenosine triphosphate;
the metabolic intermediate of the glycolysis pathway comprises one or more of glucose-6-phosphate, fructose-6-phosphate, fructose-1,6-diphosphate, 3-phosphoglyceraldehyde, dihydroxyacetone phosphate, 1,3-diphosphoglycerate, 3-phosphoglycerate, 2-phosphoglycerate, phosphoenolpyruvate (PEP), and pyruvate;
the polymer monomer capable of being converted into acetyl-coenzyme A is 3-hydroxybutyric acid.

3. The bioenergetic-active material according to claim 1, wherein the bioenergetic-active material is a polyhydroxyalkanoate having a degradation product of 3-hydroxybutyric acid.

4. The bioenergetic-active material according to claim 3, wherein the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

5. Use of the bioenergetic-active material according to claim 1 in the field of bone tissue regeneration and repair.

6. Use of the bioenergetic-active material according to claim 1 in the manufacture of a porous scaffold for bone tissue repair.

7. A porous scaffold for bone tissue repair manufactured from the bioenergetic-active material according to claim 1.

8. The method for manufacturing a 3D porous scaffold for bone tissue repair, comprising the following steps:
(1) synthesizing the bioenergetic-active material according to claim 1; and
(2) manufacturing a 3D porous scaffold for bone tissue repair in combination with a 3D printing technology.

9. The method according to claim 8, wherein the bioenergetic-active material is synthesized by a microbiological or chemical synthesis method.

10. A 3D porous scaffold for bone tissue repair manufactured by the method according to claim 8.

11. Use of a polyhydroxyalkanoate having a degradation product comprising 3-hydroxybutyric acid as a bioenergetic-active material with both bone tissue regeneration and angiogenesis functions, wherein 3-hydroxybutyric acid is, in a form of citrate, involved in bone formation via a tricarboxylic acid metabolic cycle, and 3-hydroxybutyric acid induces angiogenesis.

12. The use according to claim 11, wherein the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

13. Use of a polyhydroxyalkanoate having a degradation product comprising 3-hydroxybutyric acid in the preparation of a vascularized bone regeneration material.

14. The use according to claim 13, wherein the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

15. Use of a polyhydroxyalkanoate having a degradation product comprising 3-hydroxybutyric acid in the preparation of a large-sized bone defect repair material or a critical bone defect repair material.

16. The use according to claim 15, wherein the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).

17. A vascularized bone regeneration material, a large-sized bone defect repair material or a critical bone defect repair material, prepared from a polyhydroxyalkanoate having a degradation product comprising 3-hydroxybutyric acid.

18. The material according to claim 17, wherein the material is a porous scaffold manufactured from the polyhydroxyalkanoate having the degradation product comprising 3-hydroxybutyric acid.

19. The material according to claim 17, wherein the polyhydroxyalkanoate comprises one or more of poly(3-hydroxybutyrate-co-4-hydroxybutyrate), poly-3-hydroxybutyrate, poly(3-hydroxybutyrate-co-3-hydroxyvalerate), and poly(3-hydroxybutyrate-co-3-hydroxyhexanoate).
